(19) United States
(12) Patent Application Publication
(10) Pub. No.: US 2004/0249459 A1
(43) Pub. Date: Dec. 9, 2004

(54) Nucleus replacements with asymmetrical stiffness

(76) Inventor: Bret A. Ferree, Cincinnati, OH (US)

Correspondence Address: John G. Posa, Gifford, Krass, Groh, Sprinkle,, Anderson & Citkowski, P.C., 280 N. Old Woodward Ave., Suite 400, Birmingham, MI, 48009-5394, US

(21) Appl. No.: 10/859,010

(22) Filed: Jun. 2, 2004

Related U.S. Application Data
(60) Provisional application No. 60/475,160, filed on Jun. 2, 2003.

(51) Int. Cl. A61F 2/44; A61F 2/02

(52) U.S. Cl. 623/11.11

(57) ABSTRACT

Nucleus replacement (NR) devices are less flexible from side-to-side. In preferred embodiments, NR devices according to the invention have increased lateral, or side-to-side, stiffness to decrease the risk of extruding the NR through the holes in the annulus fibrosis (AF). That is, certain NRs according to this invention have increased lateral stiffness that prevent them from becoming narrow enough to escape through a hole or defect in the AF.

### REFERENCE TO RELATED APPLICATION

This application claims priority from U.S. Provisional Patent Application Ser. No. 60/475,160, filed Jun. 2, 2003, the entire content of which is incorporated herein by reference.

### FIELD OF THE INVENTION

This invention relates generally to artificial disc replacements (ADRs) and, in particular, to nucleus replacements with increased lateral stiffness.

### BACKGROUND OF THE INVENTION

According to human anatomy, spinal function is dependent upon the intervertebral disc and the facet joints. In a sense, the annulus fibrosis, nucleus pulpous, and the facet joints form the legs of a three-legged stool.

To restore disc height resulting, for example, from degenerative disease, prosthetic discs are used to replace only the nucleus pulpous. Reference is made to U.S. Pat. No. 6,419,704, which discusses spinal anatomy, spinal physiology, disc degeneration, surgical and non-surgical treatments of disc disease, and the advantages of prosthetic disc replacement.

The annulus is formed of 10 to 60 fibrous bands which serve to control vertebral motion. One half of the bands tighten to check motion when the vertebra above or below the disc are turned in either direction. Restoring disc height returns tension to the annular noted in the prosthetic disc patent application. In addition, restoring annular tension decreases annular protrusion into the spinal canal or neural foramen. Thus, decreasing annular protrusion may eliminate pressure on the spinal cord or nerve roots.

At times the rotational, translational, and axial compression forces exceed the strength of the annular fibers. The excessive forces tear the annular fibers. A single event can tear one band to all the bands. Subsequent tears can connect to previous tears of a few bands resulting in a hole through the entire annulus fibrosis. Holes through the entire annulus fibrosis can result in extrusion of the nucleus pulpous. Extrusion of the nucleus pulpous is referred to as a “herniated disc.” Disc herniation can result in back pan, neck pain, arm pain, leg pain, nerve or spinal cord injury, or a combination of the above.

Since the annulus is innervated with pain fibers, acute annular tears without herniation of the nucleus can be painful. Unfortunately, the annular tears often do not heal completely. The chronic tears can result in neck pain, back pain, shoulder pain, buttock pain, or thigh pain. The chronic tears weaken the annulus fibrosis predisposing the disc to herniation or additional annular tears. My U.S. Pat. No. 6,340,369, entitled “Treating Degenerative Disc Disease With Harvested Disc Cells and Analogies of the Extracellular Matrix,” and U.S. Pat. No. 6,419,704, entitled “Artificial Intervertebral Disc Replacement Method And Apparatus” describe methods and apparatus for occluding annular defects.

Prosthetic replacement of the nucleus pulpous alone risks future problems arising from annular tears. Patients may continue to complain of pain from the stresses placed onto the weakened annulus. Secondly, tears of the annulus could result in extrusion of the prosthetic nucleus. In addition, remaining nucleus pulpous could herniate through annular tears.

Some prosthetic disc designs attempt to replace nucleus and annular functions. In general, these designs attach the prosthetic disc to the vertebrae. Many of the techniques in this area attach the prosthetic disc to the end plates of the vertebrae with screws, spikes, flanges, or porous surfaces for bone ingrowth. My U.S. Pat. Nos. 6,245,107 and 6,419,704 describe methods and devices to assist the annulus in retaining remaining nucleus pulpous and a prosthetic nucleus. The entire contents of these applications are incorporated herein by reference.

Nucleus replacement (NR) devices are often used to replace or augment the nucleus pulposus (NP) of the intervertebral disc. The flexible devices cushion the loads applied to the disc. The devices must be flexible in the direction from the top of the device to the bottom of the device.

Prior-art NRs are generally as stiff from top to bottom as they are from side to side. Prior-art NRs often enlarge by imbibing fluid or cure in-situ in an attempt to prevent the NR from extruding from the hole they were inserted through. Unless the NR is placed directly in the center of the disc space, the pressure within the disc space facilitates extrusion of the NR through the hole in the AF.

Flexible prior-art NRs change shape in response to the pressure applied to them. Prior-art NRs can become narrow enough to escape through the hole in the AF, by the forces applied to the front of the NR and the AF on either side of the hole in the AF. A need therefore remains for NR devices which function in a natural way but which are less prone to extrusion or misalignment.

### SUMMARY OF THE INVENTION

This invention improves upon the prior art by providing nucleus replacement (NR) devices that are broadly less flexible from side-to-side. In preferred embodiments, NR devices according to the invention have increased lateral, or side-to-side, stiffness to decrease the risk of extruding the NR through the holes in the annulus fibrosis (AF). That is, certain NRs according to this invention have increased lateral stiffness that prevent them from becoming narrow enough to escape through a hole or defect in the AF.

### BRIEF DESCRIPTION OF THE DRAWINGS

FIG. 1A is an axial cross-section of a NR according to the present invention;

FIG. 1B is an axial cross-section of a disc and the first step in inserting the device drawn in FIG. 1A;

FIG. 1C is an axial cross-section of a disc and the final position of the device drawn in FIG. 1A;

FIG. 2A is an axial cross-section through an alternative embodiment of the nucleus replacement (NR);

FIG. 2B is a view of the side of the embodiment of the NR drawn in FIG. 2A;

FIG. 3A is an axial cross-section of an alternative embodiment of the NR drawn in FIG. 2A;

FIG. 3B is an axial cross-section of a disc and the embodiment of the NR drawn in FIG. 3A;

FIG. 3C is an axial cross-section of a disc and the embodiment of the NR drawn in FIG. 3B;

FIG. 4A is an axial cross-section of an alternative embodiment of the NR;

FIG. 4B is an axial cross-section of the second shape of the embodiment of the NR drawn in FIG. 4A;

FIG. 5A is an axial cross-section of the embodiment of the NR drawn in FIG. 4A;

FIG. 5B is an axial cross-section of the second shape of the NR drawn in FIG. 5A;

FIG. 6A is an axial cross-section of another embodiment of the device;

FIG. 6B is an axial cross-section of the embodiment of the device drawn in FIG. 6A in its second, wide shape;

FIG. 7A is an axial cross-section of an alternative embodiment of the NR;

FIG. 7B is an axial cross-section of the embodiment of the NR drawn in FIG. 7A in its second, wider shape;

FIG. 7C is an axial cross-section of an alternative embodiment of the NR drawn in FIG. 7A;

FIG. 8A is an axial cross-section through another embodiment of the present invention;

FIG. 8B is an axial cross-section through the embodiment of the device drawn in FIG. 8A, in its second, wide position;

FIG. 9A is an axial cross-section through an alternative embodiment of the present invention;

FIG. 9B is an axial cross-section through the embodiment of the device drawn in FIG. 9A;

FIG. 9C is an axial cross-section of the embodiment of the device drawn in FIG. 9B, after inflating the NR;

FIG. 10A is an axial cross-section of an alternative embodiment of the NR drawn in FIG. 1A;

FIG. 10B is an axial cross-section of the embodiment of the NR drawn in FIG. 10A, rotated 90 degrees;

FIG. 10C is a sagittal cross-section of the embodiment of the NR drawn in FIG. 10A;

FIG. 10D is a sagittal cross-section through an alternative embodiment of the NR drawn in FIG. 10C;

FIG. 11A is an axial cross-section through another embodiment of the present invention;

FIG. 11B is an axial cross-section through the embodiment of the device drawn in FIG. 11A, after injection of the in-situ curing material;

FIG. 12A is an axial cross-section of an intervertebral disc after removal of a portion of the NP;

FIG. 12B is an oblique view of an enclosure component of a NR;

FIG. 12C is an axial cross-section of the disc and a collapsed enclosure component;

FIG. 12D is view of the top of another embodiment of a device with increased lateral stiffness;

FIG. 12E is an axial cross-section of the disc and an axial cross-section of the embodiment of the device drawn in FIGS. 12A-12D;

FIG. 12F is an axial cross-section of a disc and the embodiment of the invention drawn in FIG. 12E;

FIG. 13A is a view of the top of an alternative component, that may placed into the enclosure drawn in FIG. 12B;

FIG. 13B is a view of the top of the component drawn in FIG. 13A;

FIG. 13C is the view of the top of a component similar to that shown in FIG. 13A;

FIG. 13D is a view of the side of the component drawn in FIG. 13A;

FIG. 14A is a view of the top of the component drawn in FIG. 13C;

FIG. 14B is a view of the side of the component drawn in FIG. 14A;

FIG. 15A is a view of the top of an alternative embodiment of the stiff component drawn in FIG. 13C;

FIG. 15B is a view of the top of the component drawn in FIG. 15A;

FIG. 15C is the view of the top of the component drawn in FIG. 15B;

FIG. 15D is a view of the top of an optional second cross member;

FIG. 15E is a view of the top of the component drawn in FIG. 15B;

FIG. 16A is the view of an alternative embodiment of the stiff component drawn in FIG. 15A;

FIG. 16B is a lateral view of the device drawn in FIG. 16A;

FIG. 16C is a view of the sides of the tips of the arms of the device drawn in FIG. 16A;

FIG. 16D is a cross-section through the spring loaded axle of the device drawn in FIG. 16A;

FIG. 16E is a cross-section through the spring loaded axle of the device drawn in FIG. 16D;

FIG. 16F is a view of the top of the stiff component drawn in FIG. 16A;

FIG. 16G is an axial view of the disc, a NR enclosure, and the embodiment of the stiff component drawn in FIG. 16A;

FIG. 16H is an axial view of the disc and the embodiment of the stiff component drawn in FIG. 16A;

FIG. 17A is an axial cross-section of a disc, an enclosure, and a component of an alternative stiff device;

FIG. 17B is an axial cross-section of a disc, an enclosure, and the embodiment of the stiff component drawn in FIG. 17A;

FIG. 17C is an axial cross-section of a disc, an enclosure, and the embodiment of the stiff component drawn in FIG. 17B;

FIG. 17D is a lateral view of the embodiment of the stiff components drawn in FIG. 17C;

FIG. 18A is a view of the top of another embodiment of the stiff component;

FIG. 18B is a view of the top of the embodiment of the stiff component drawn in FIG. 18A;

FIG. 19A is the view of the top of an alternative embodiment of the stiff component drawn in FIG. 18A;

FIG. 19B is a cross-section of the embodiment of the device drawn in FIG. 19A;

FIG. 19C is view of the top of the embodiment of the device drawn in FIG. 19A;

FIG. 19D is a cross-section of the embodiment of the stiff component drawn in FIG. 19C;

FIG. 20A is a view of the top of an alternative stiff component;

FIG. 20B is a view of the top of the embodiment of the component drawn in FIG. 20A;

FIG. 21A is a view of the top of an assembled cushion component;

FIG. 21B is a view of the top of the cushion components drawn in FIG. 21A;

FIG. 22A is an exploded view of an alternative embodiment of the present invention;

FIG. 22B is a view of the top of the cage drawn in FIG. 22A;

FIG. 23A is an axial cross-section of an exploded, alternative embodiment of a NR device according to the present invention;

FIG. 23B is an axial cross-section of an assembled embodiment of the device drawn in FIG. 23A;

FIG. 24A is a view of the top of an alternative embodiment of a stiff component;

FIG. 24B is a view of the top the component drawn in FIG. 24A;

FIG. 24C is an oblique view of a cap component;

FIG. 24D is a lateral view of the device drawn in FIG. 24A;

FIG. 25A is a view of the top of an alternative embodiment of the stiff component;

FIG. 25B is a view of the top of the embodiment of the stiff component drawn in FIG. 25A;

FIG. 25C is a view of the top of an alternative embodiment of the component drawn in FIG. 25A;

FIG. 25D is a view of the top of the embodiment of the device drawn in FIG. 25C;

FIG. 25E is a view of the top of an alternative embodiment of the stiff component drawn in FIG. 25C;

FIG. 25F is a view of the top of the embodiment of the device drawn in FIG. 25E;

FIG. 25G is a view of the top of an alternative embodiment of the stiff component drawn in FIG. 25E;

FIG. 25H is a view of the top of the embodiment of the device drawn in FIG. 25G;

FIG. 25I is a view of the top of an alternative embodiment of the device drawn in FIG. 25G;

FIG. 25J is a view of the top of the device drawn in FIG. 251;

FIG. 26A is a lateral view of another embodiment of the stiff component;

FIG. 26B is a lateral view of the stiff device drawn in FIG. 26A;

FIG. 26C is a cross-section of the device drawn in FIG. 26B;

FIG. 26D is an axial cross-section of the disc, an enclosure, and the stiff device drawn in FIG. 26B;

FIG. 26E is an axial cross-section of the disc, the enclosure, and the embodiment of the stiff device drawn in FIG. 26D;

FIG. 26F is a lateral view of the stiff component drawn in FIG. 26A and compression band;

FIG. 26G is a lateral view of the stiff component drawn in FIG. 26F and the compression band;

FIG. 27A is an axial cross-section and an alternative embodiment of the device drawn in FIG. 26E;

FIG. 27B is a sagittal cross-section of the device drawn in FIG. 27A;

FIG. 27C is a sagittal cross-section of the device drawn in FIG. 27B;

FIG. 27D is cross-section of the reinforced material around the embodiment of the stiff device drawn in FIG. 27A and the stiff device;

FIG. 27E is an exploded partial cross-section of the embodiment of the device drawn in FIG. 27A;

FIG. 27F is an oblique view of the optional cover component drawn in FIG. 27A; and

FIG. 27G is a partial axial cross-section of the disc and an alternative embodiment of the invention drawn in FIG. 27A.

### DETAILED DESCRIPTION OF THE INVENTION

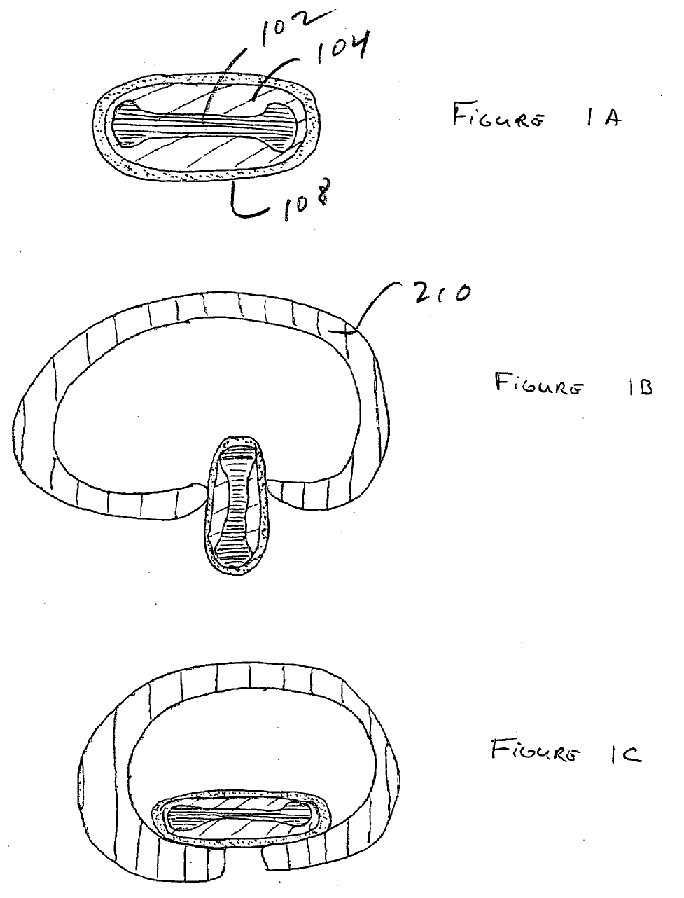

FIG. 1A is an axial cross-section of a nucleus replacement (NR) according to the invention. An elongated member 102 of increased stiffness is surrounded by a second, less stiff material 104. For example, a stiff metal or polymer component could be surrounded by a hydrogel or polyurethane component. Both components could be surrounded by a carcass 108 as described in my U.S. Pat. No. 6,419,704, incorporated herein by reference.

FIG. 1B is an axial cross-section of a disc and the first step in inserting the device drawn in FIG. 1A. The device is inserted through a hole in the AF. The AF is represented by the area of the drawing with vertical lines.

FIG. 1C is an axial cross-section of a disc and the final position of the device drawn in FIG. 1A. The NR device has been rotated to increase the left to right stiffness of the NR. The increased lateral stiffness of the NR helps prevent the NR from changing to a shape that allows the NR to extrude through the hole in the AF. Although FIG. 1C shows the device at a certain size and shape relative to the disc into which it is inserted, it will be appreciated that the device may be differently shaped, smaller, or large enough to consume the entire disc space.

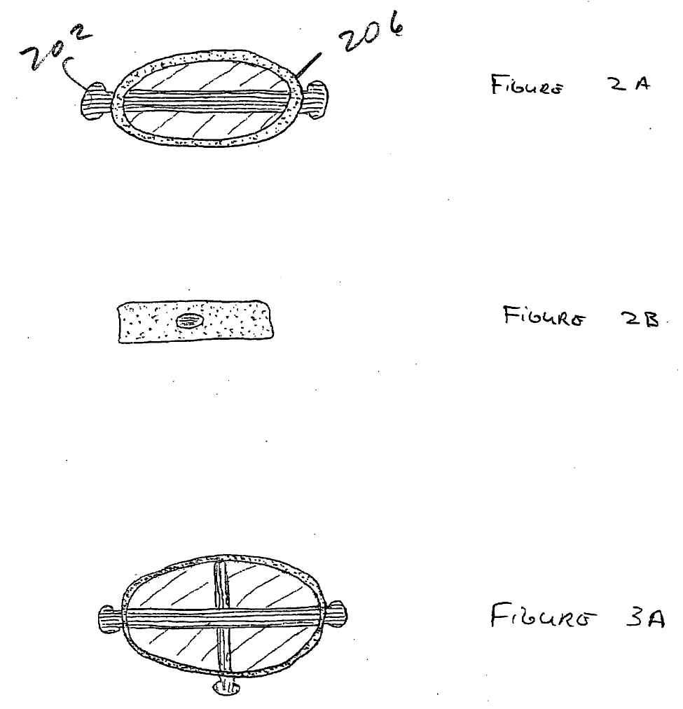

FIG. 2A is an axial cross-section through an alternative embodiment of the NR. FIG. 2B is a view of the side of the embodiment of the NR drawn in FIG. 2A. The member 202 with increased lateral stiffness extends through a hole in the carcass 206 of the NR. The elastic carcass can expand and contract over the member with increased lateral stiffness.

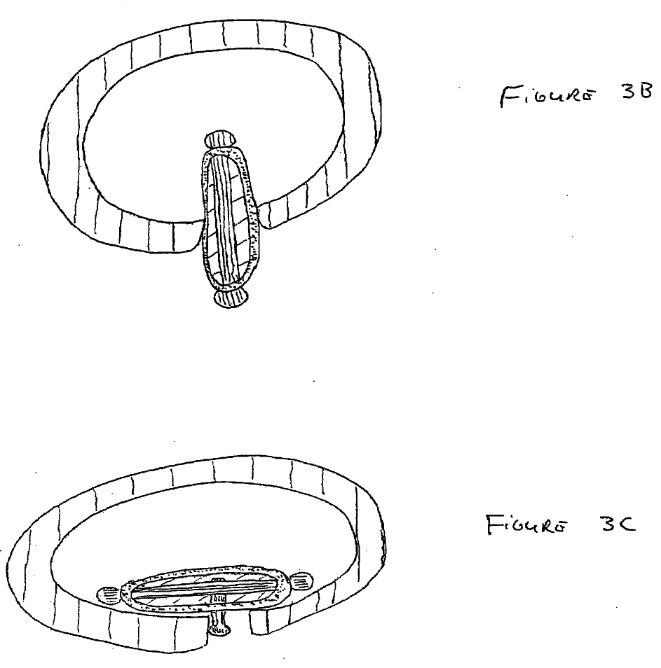

FIG. 3A is an axial cross-section of an alternative embodiment of the NR drawn in FIG. 2A. A second member with increased lateral stiffness can be added to the NR after the NR is rotated 90 degrees. The second stiff member helps prevent the NR from extruding through the hole in the AF, in the event the NR rotates 90 degrees and returns to its first position during the first step of insertion. FIG. 3B is an axial cross-section of a disc and the embodiment of the NR drawn in FIG. 3A, during the first step of insertion.

FIG. 3C is an axial cross-section of a disc and the embodiment of the NR drawn in FIG. 3B, during the second step in the insertion of the device. The NR has been rotated 90 degrees after insertion into the disc space. The second stiff member is shown during its insertion into the NR.

FIG. 4A is an axial cross-section of an alternative embodiment of the NR. The stiff inner member 402 has shape-memory properties. Alternatively, the stiff member may have spring-like properties. The stiff member is drawn in its first, narrow shape. Lateral pressure can be applied to the NR that contains a stiff spring member.

FIG. 4B is an axial cross-section of the second shape of the embodiment of the NR drawn in FIG. 4A. The projections from the sides of the stiff member are drawn in an extended position.

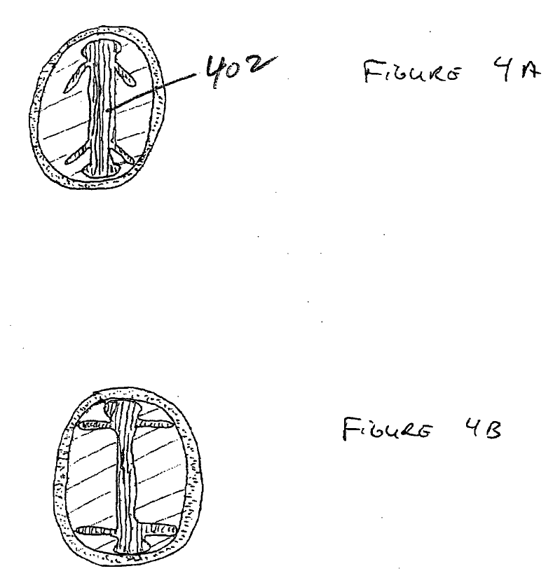

FIG. 5A is an axial cross-section of the embodiment of the NR drawn in FIG. 4A. The stiff inner member is drawn in its first, narrow shape. FIG. 5B is an axial cross-section of the second shape of the NR drawn in FIG. 5A. The projections from the sides of the stiff member are drawn in an extended position. The projects can lengthen by shape memory technology. Alternatively, the projections can lengthen by removing lateral force applied to the sides of a spring containing NR.

FIG. 6A is an axial cross-section of another embodiment of the device. The stiff inner member 602 is drawn in its first, narrow shape. FIG. 6B is an axial cross-section of the embodiment of the device drawn in FIG. 6A in its second, wide shape. Shape memory technology or a spring component may be used to achieve the shape change.

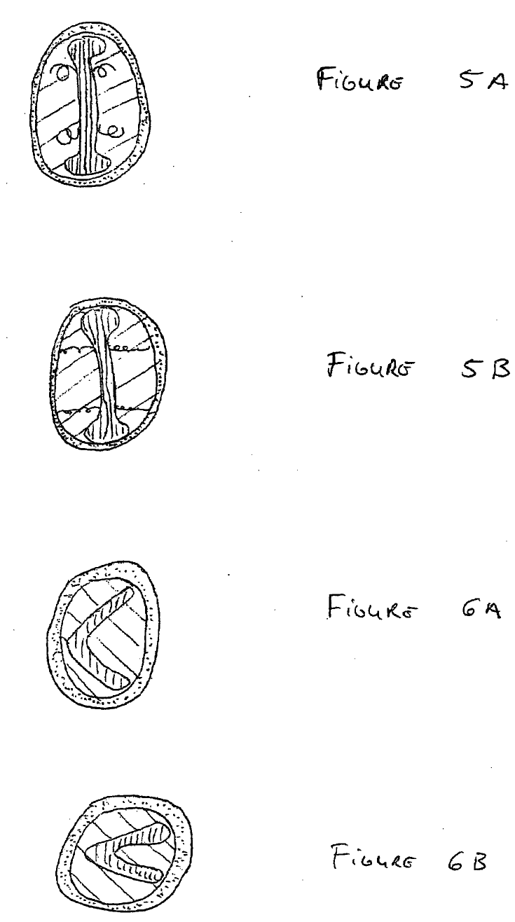

FIG. 7A is an axial cross-section of an alternative embodiment of the NR. Two members with increased lateral stiffness are drawn within the NR. The stiff members area drawn in a narrow, first position. FIG. 7B is an axial cross-section of the embodiment of the NR drawn in FIG. 7A in its second, wider shape. The crossed stiff members are scissored open. External pressure can be applied to the NR to encourage the stiff members to change their position. The stiff members may have a mechanism that locks them in the second shape. For example, the stiff members can be connected by an axle. The stiff members could be biased by a spring pulling them together. The stiff members may have locking slots that receive and lock the opposite stiff member in the wide position.

FIG. 7C is an axial cross-section of an alternative embodiment of the NR drawn in FIG. 7A. Cushioning components with properties could be used to deploy the arms of the stiff component. For example, the cushioning material represented by the area of the drawing with crossed diagonal lines could be a hydrogel or other substance to imbibe fluid, thus forcing the arms of the stiff component into an expanded position.

FIG. 8A is an axial cross-section through another embodiment of the device. A stiff cross member is drawn in its first, narrow position. FIG. 8B is an axial cross-section through the embodiment of the device drawn in FIG. 8A, in its second, wide position. The stiff cross member can be spring biased to extend as pressure is relieved from the sides of the NR. Alternatively, shape memory technology can be used to achieve the shape change. In either case, the NR may have a mechanism to lock the cross member in its extended position. For example, the cross member may fit into a slot in the NR.

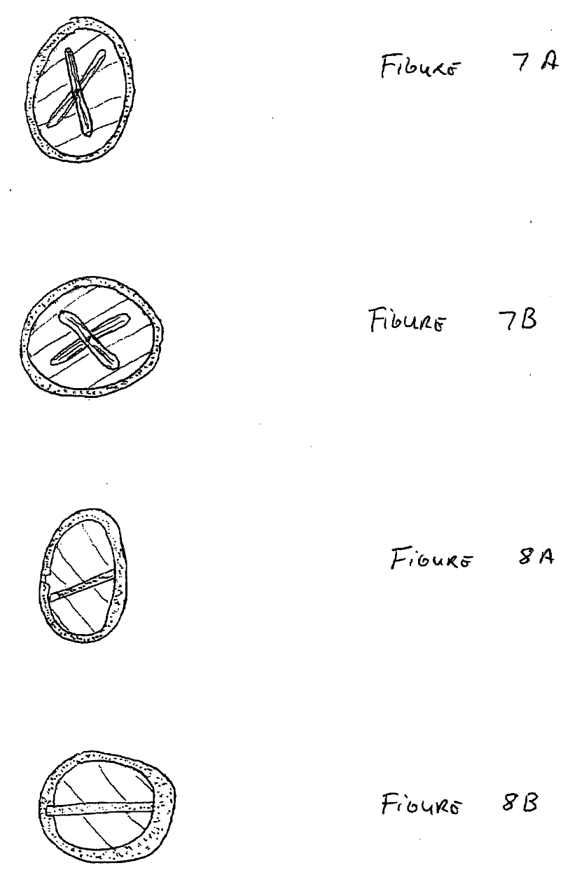

FIG. 9A is an axial cross-section through an alternative embodiment of the device. The stiff component is contained within a deflated carcass (dotted area of the drawing). The projections from the stiff component are drawn in their first, narrow position. FIG. 9B is an axial cross-section through the embodiment of the device drawn in FIG. 9A. The projections are drawn in their second, extended position. Shape memory technology can be used to achieve the shape change. Alternatively, a material with spring properties could be used to achieve the shape change. FIG. 9C is an axial cross-section of the embodiment of the device drawn in FIG. 9B, after inflating the NR. The NR could be inflated with in-situ curing polymers.

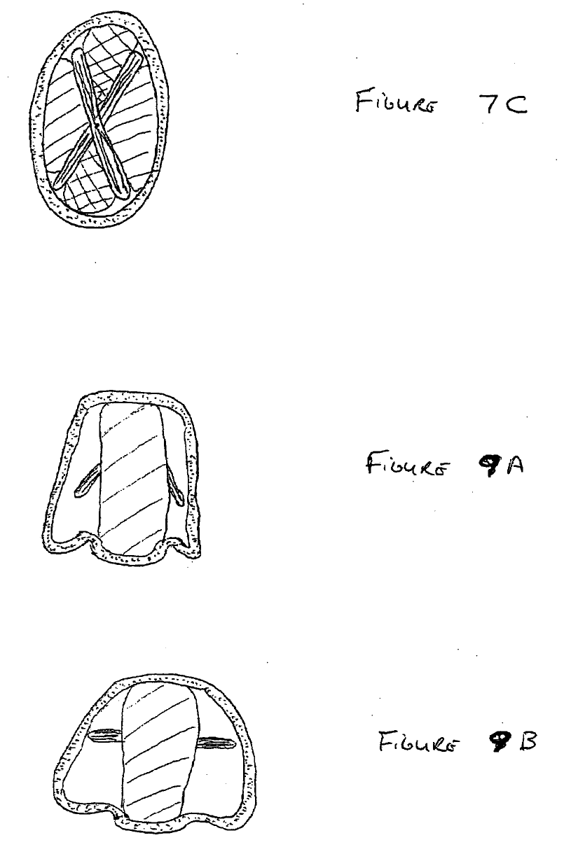

FIG. 10A is an axial cross-section of an alternative embodiment of the NR drawn in FIG. 1A. The NR contains two stiff components 120, 122. More than two stiff components can be used. FIG. 10B is an axial cross-section of the embodiment of the NR drawn in FIG. 10A, rotated 90 degrees. FIG. 10C is a sagittal cross-section of the embodiment of the NR drawn in FIG. 10A. The stiff members are surrounded by the cushioning component.

FIG. 10D is a sagittal cross-section through an alternative embodiment of the NR drawn in FIG. 10C. The stiff components are located above and below the cushion component. The stiff components can sit between the carcass and the cushion component. Alternatively, the stiff components may be contained within the carcass. Stiff components can be used on the top, the bottom, or both the top and bottom of the NR. The carcass of the NR may have holes in the top and bottom of the carcass.

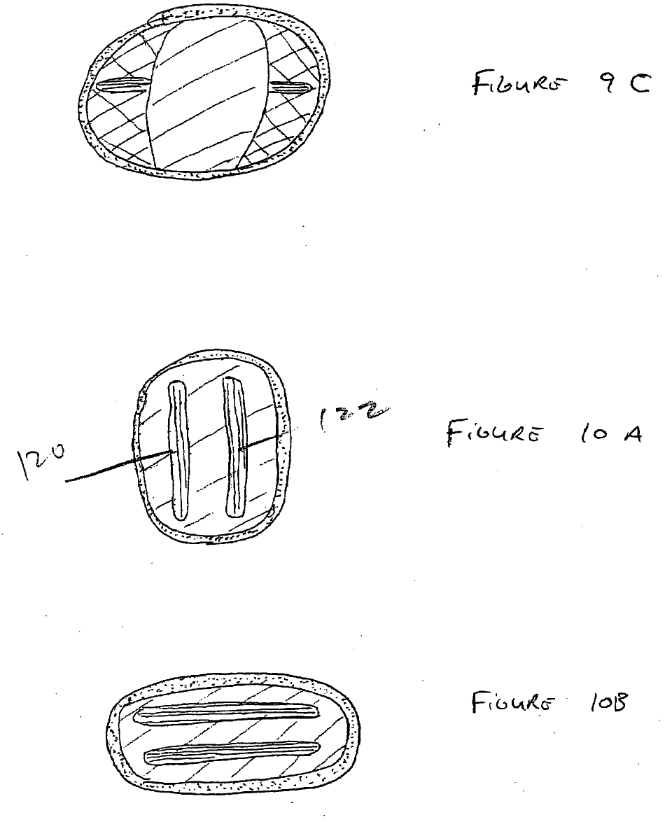

FIG. 11A is an axial cross-section through another embodiment of the device. The stiff component indicated at 1102. The area 1104 within the cushion component represents space for injecting an in-situ curing, situ material. The in-situ curing material is preferably injected after the NR is inserted. FIG. 11B is an axial cross-section through the embodiment of the device drawn in FIG. 11A, after injection of the in-situ curing material.

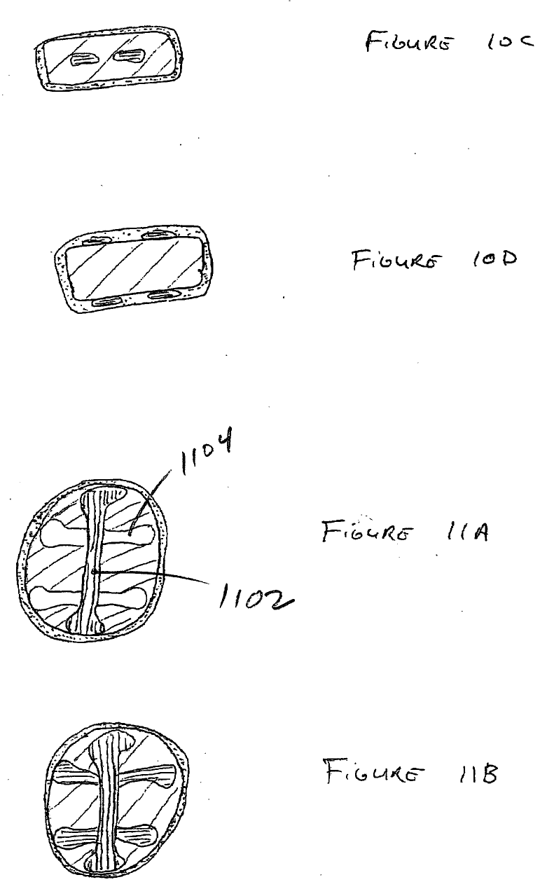

FIG. 12A is an axial cross-section of an intervertebral disc after removal of a portion of the NP. The defect 1202 in the AF is seen on the bottom of the drawing. A portion 1204 of the NP has been retained. FIG. 12B is an oblique view of an enclosure component of a NR. The enclosure has arms 1206, 1208 that extend from the sides of the device. An opening is seen between the arms of the device. The enclosure may be constructed from a flexible synthetic or natural material. Synthetic materials include biocompatible polymers or fiber-like materials including Gortex or Dacron. The device may be impermeable or porous to allow fluids to diffuse into and out of the device.

FIG. 12C is an axial cross-section of the disc and a collapsed enclosure component. The collapsed enclosure is placed into the disc through a hole in the AF. FIG. 12D is view of the top of another embodiment of a device with increased lateral stiffness. The device is made of a shape memory material, for example Nitinol. The long narrow first shape of the device facilitates placement into the NR enclosure component through the defect in the AF and the opening in the enclosure component.

FIG. 12E is an axial cross-section of the disc and an axial cross-section of the embodiment of the device drawn in FIGS. 12A-12D. The enclosure component lies along the inner surface of the AF. The arms of the enclosure extend along the outer surface of the AF on either side of the AF. The component that increases lateral stiffness of the NR is seen inside the enclosure. The stiffer component has assumed a second shape. The coiled wires with the stiffer component lengthened in response to a change in temperature. Lengthening of the coiled wires increases the lateral dimension of the component. The stiffer component now extends beyond the edges of the defect in the AF. The stiffness provide by the shape memory component prevent the NR from collapsing and extruding through the defect in the AF. A cushion component is placed into the enclosure before closing the opening in the enclosure. Cushion materials may be synthetic and/or natural. For example, morselized nucleus and/or annulus from the same disc may be used for this purpose, though other biocompatible materials may alternatively be used. In addition to autograft nucleus pulposus, the device may be filled with allograft nucleus pulposus, xenograft nucleus pulposus, other tissue and/or synthetic materials such as hydrogels or elastomers. In situ curing materials may also be used.

FIG. 12F is an axial cross-section of a disc and the embodiment of the invention drawn in FIG. 12E. The cushion and stiff components can be seen within the enclosure. The enclosure has been attached to the AF. For example, staples could be passed through the arms of the enclosure, the AF, and a wall of the enclosure that lies in the disc space. Alternatively plastic devices similar to those used to attach price tags to garments could be used to attach the device to the AF. The opening in the enclosure has also been closed with staples. The opening of the NR enclosure need not be closed when using certain in-situ curing materials such as foam polyurethane.

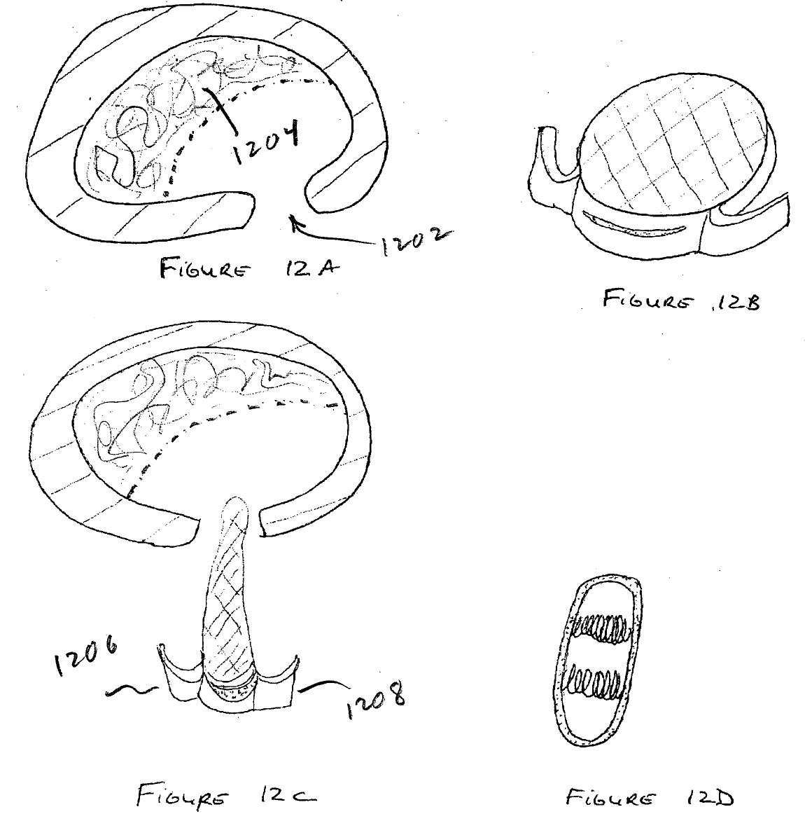

FIG. 13A is a view of the top of an alternative component, that may placed into the enclosure drawn in FIG. 12B, to increase stiffness of the NR. The device is preferably constructed of a shape-memory material. A polymer material, such as stearle methacrylate, could be used in a solid device. A metal, such as Nitinol, could be used in a wire device. The device is drawn in its narrow first shape. The narrow shape facilitates insertion into the NR enclosure, through the defect in the AF.

FIG. 13B is a view of the top of the component drawn in FIG. 13A. The component is drawn in its second, expanded shape. The component changes to its second shape after it is placed in the enclosure that lies in the disc space. The shape change may be caused by a change in temperature, or other stimuli known to effect shape change materials.

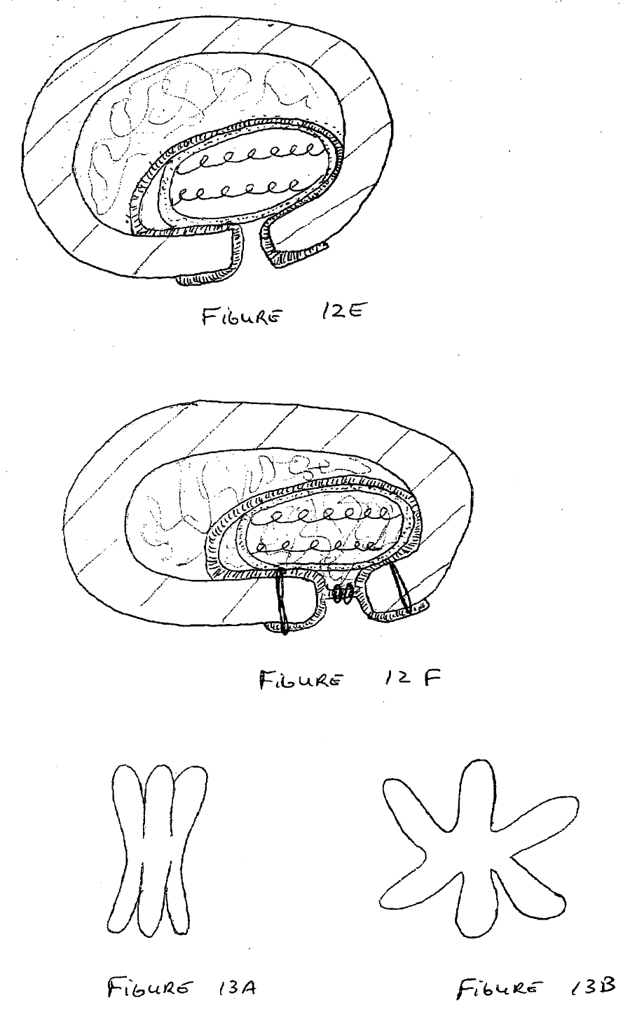

FIG. 13C is the view of the top of a component similar to that drawn in FIG. 13A. The component is drawn in its first shape. FIG. 13D is a view of the side of the component drawn in FIG. 13A. The arms of the component lie upon one another to facilitate insertion through the defect in the AF. FIG. 14A is a view of the top of the component drawn in FIG. 13C. The component is drawn in its second shape. FIG. 14B is a view of the side of the component drawn in FIG. 14A. The arms of the component have fanned out to increase the lateral stiffness of the NR.

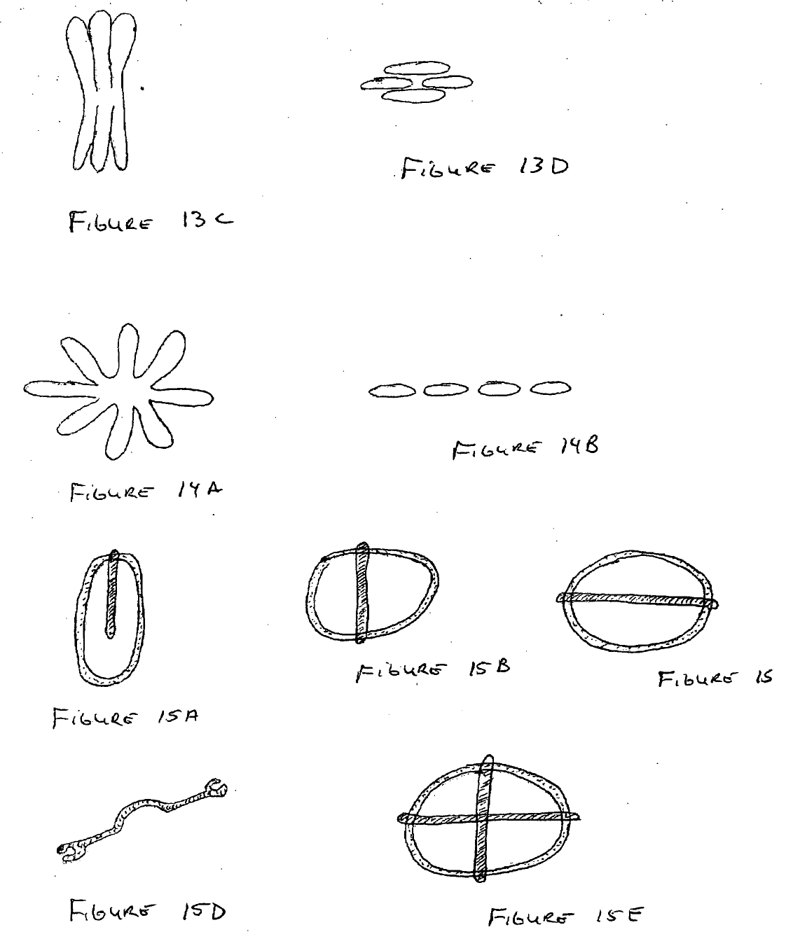

FIG. 15A is a view of the top of an alternative embodiment of the stiff component drawn in FIG. 13C. The component is drawn in its first, narrow shape. The device is made of material with spring-like properties. FIG. 15B is a view of the top of the component drawn in FIG. 15A. The spring-like band or hoop expands after it is placed into the NR enclosure. The center component of the device fastens to both sides of the band. The center component, like that drawn in FIG. 8B, increases the stiffness of the band.

FIG. 15C is the view of the top of the component drawn in FIG. 15B. The component has been rotated 90 degrees. The component is rotated 90 degrees after it is placed into the enclosure, and after both ends of the center component are fastened to the band. FIG. 15D is a view of the top of an optional second cross member.

FIG. 15E is a view of the top of the component drawn in FIG. 15B. The second cross member is attached to the hoop. The use of two cross members maintains the lateral stiffness of the component even if the component rotates in the disc space. The NR enclosure is filled with a cushion component before or after placement of the stiff component.

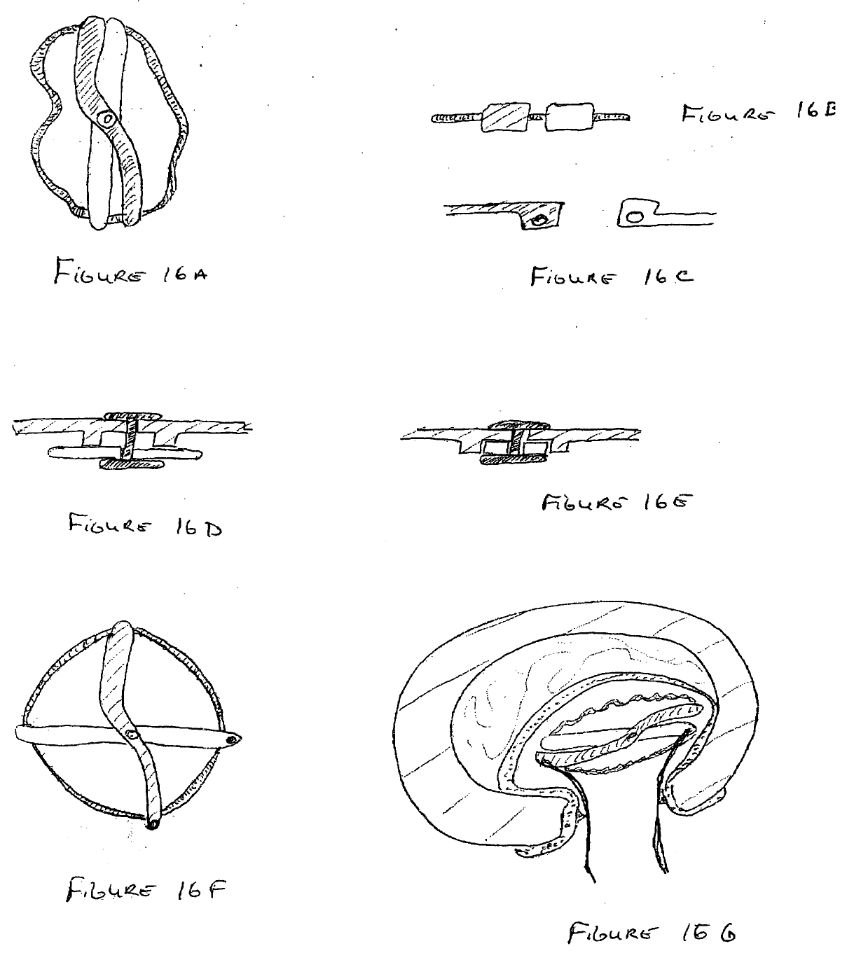

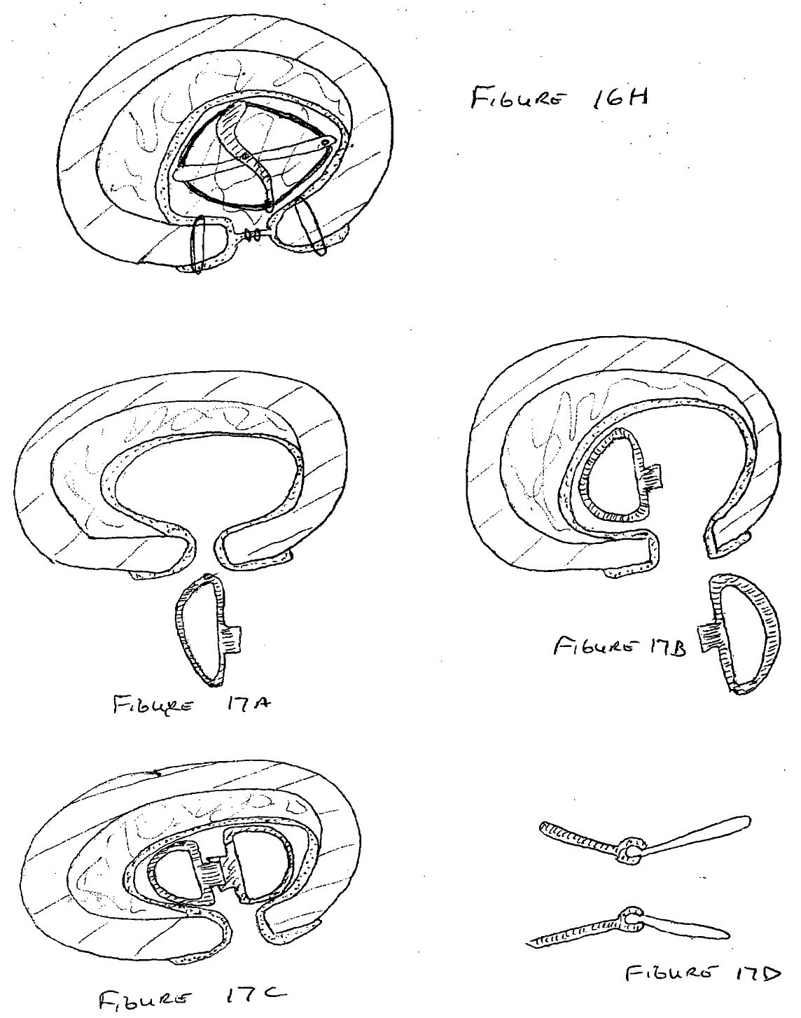

FIG. 16A is the view of an alternative embodiment of the stiff component drawn in FIG. 15A. The device has scissor-like arms that rotate around a spring-loaded axle. A cable hoop extends through the tips of the arms of the device. The device is drawn in its narrow first shape.

FIG. 16B is a lateral view of the device drawn in FIG. 16A. The cable can be seen coursing through holes in the arms of the device. FIG. 16C is a view of the sides of the tips of the arms of the device drawn in FIG. 16A. The holes in the arms receive the cable hoop. FIG. 16D is a cross-section through the spring loaded axle of the device drawn in FIG. 16A. The arms of the scissor components are drawn in their unlocked position. The scissor components rotate around the spring loaded axle while in their unlocked position.

FIG. 16E is a cross-section through the spring loaded axle of the device drawn in FIG. 16D. The arms of the scissor components are drawn in their locked position. The spring-loaded axle pulls the first scissor component into a space in the second scissor component.

FIG. 16F is a view of the top of the stiff component drawn in FIG. 16A. The component is drawn in its expanded shape. The device is moved to the expanded shape after the device is inserted into the NR enclosure. The cable hoop increases the surface area of the device. The increased surface area of the component reduces the probability of the stiff component eroding through the NR enclosure. Holes can be seen in the ends of the scissor components. Strings can be placed through the holes. The strings can be used to help rotate and lock the scissor components. Other tools such as angled probes, pliers, and distractors can be used to help rotate the components. A bead could be affixed to the cable hoop to help rotate the scissor components.

FIG. 16G is an axial view of the disc, a NR enclosure, and the embodiment of the stiff component drawn in FIG. 16A. The stiff component was rotated 90 degrees after placement of the component in the enclosure. Strings are seen passing through the defect in the AF. Tension on the strings rotates and locks the scissor components. The strings may be removed after locking the components.

FIG. 16H is an axial view of the disc and the embodiment of the stiff component drawn in FIG. 16A. The stiff component is in its locked position. Cushion material is seen within the NR enclosure. The enclosure has been attached to the AF. The opening in the enclosure has been staple closed. Other mechanisms can be sued to close the opening in the enclosure. For example, Velcro could be used to hold a flap in the enclosure closed.

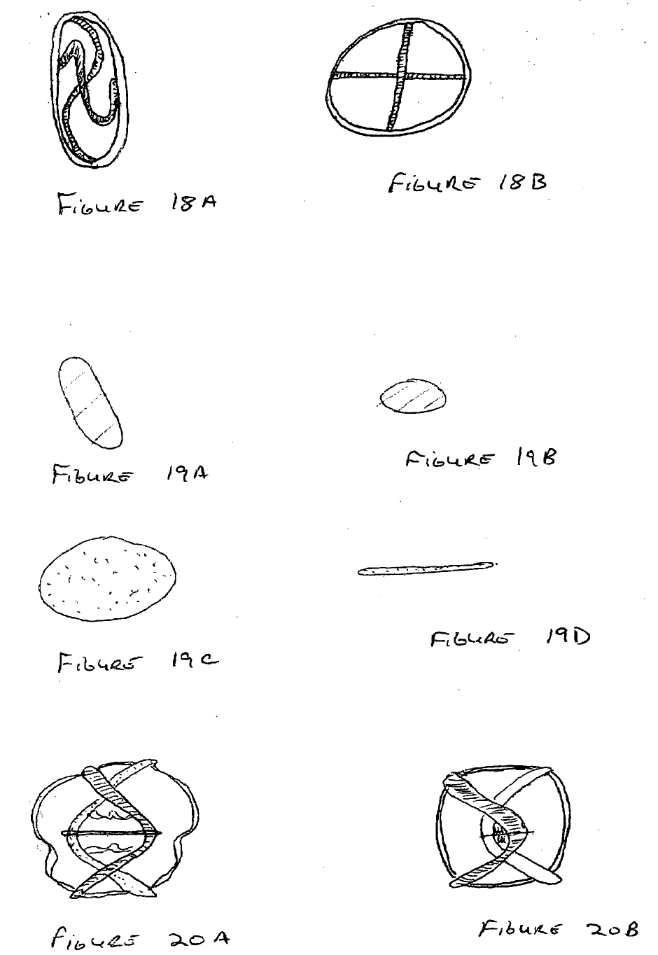

FIG. 17A is an axial cross-section of a disc, an enclosure, and a component of an alternative stiff device. The stiff component is passed through the defect in the AF. FIG. 17B is an axial cross-section of a disc, an enclosure, and the embodiment of the stiff component drawn in FIG. 17A. The first stiff component is seen within the enclosure. A second stiff component is placed into the enclosure. The stiff components are assembled within the enclosure. The devices could be fastened using shape memory technology. For example, the components could be made of Nitinol.

FIG. 17C is an axial cross-section of a disc, an enclosure, and the embodiment of the stiff component drawn in FIG. 17B. The stiff components have been fastened together. More than two components could be assembled in the enclosure. FIG. 17D is a lateral view of the embodiment of the stiff components drawn in FIG. 17C. The components may rotate at their attachment site.

FIG. 18A is a view of the top of another embodiment of the stiff component. The component is drawn in its narrow shape. The compressed device has spring or shape memory properties. FIG. 18B is a view of the top of the embodiment of the stiff component drawn in FIG. 18A. The component is drawn in its expanded shape. Cross members are used to increase the stiffness of the component. The component changes shapes after the compressed component is released in the enclosure.

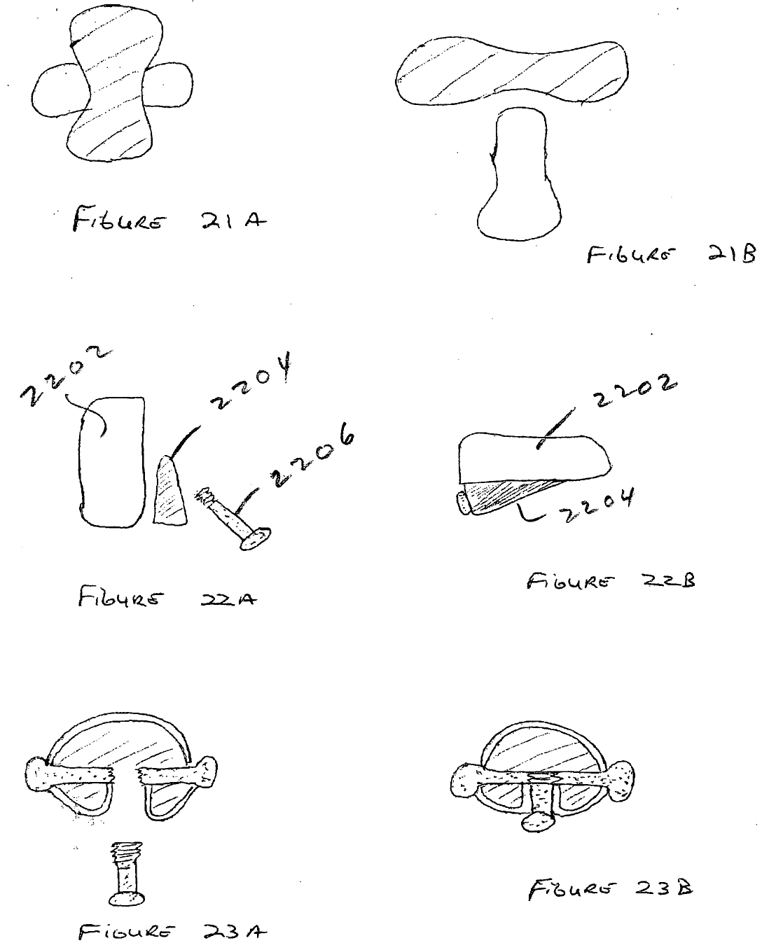

FIG. 19A is the view of the top of an alternative embodiment of the stiff component drawn in FIG. 18A. The shape memory, polymer device is drawn in its first, narrow shape. FIG. 19B is a cross-section of the embodiment of the device drawn in FIG. 19A. FIG. 19C is view of the top of the embodiment of the device drawn in FIG. 19A. The component is drawn in its expanded shape. The component assumes the expanded shape after it is placed into the enclosure. Temperature change, or fluid could cause the material to change shape. FIG. 19D is a cross-section of the embodiment of the stiff component drawn in FIG. 19C.

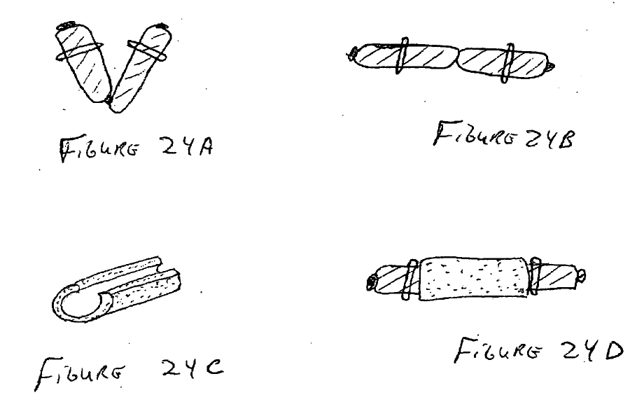

FIG. 20A is a view of the top of an alternative stiff component. The component is drawn in its narrow first shape. A cable hoop passes through the arms of the C-shaped components. The C-shaped components are connected by shape memory wires. FIG. 20B is a view of the top of the embodiment of the component drawn in FIG. 20A. The device is drawn in its second, expanded shape. The shape memory wires shorten to effect the shape change.

FIG. 21A is a view of the top of an assembled cushion component. The device is assembled within the disc space or within a NR enclosure. FIG. 21B is a view of the top of the cushion components drawn in FIG. 21A. The first component is rotated 90 degrees after placement of the device in the disc or enclosure. The second cushion component is placed through a hole in the first component. Swelling of the second component in the hole of the first component holds the components together. Alternative fastening mechanisms may be used to hold the components together.

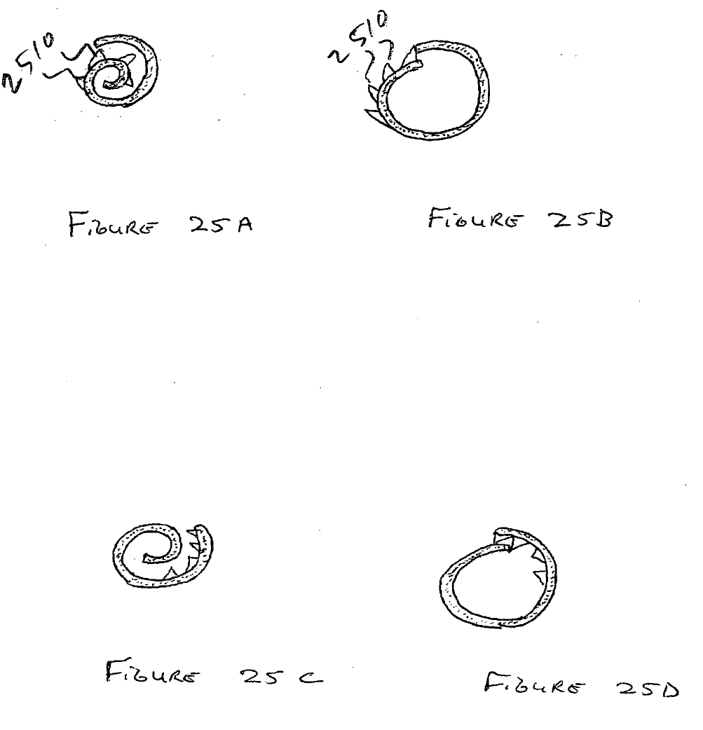

FIG. 22A is an exploded view of an alternative embodiment of the invention. Rectangle 2202 represents a fusion cage. FIG. 22B is a view of the top of the cage drawn in FIG. 22A. A second component 2204 is fastened to the cage after the cage is placed in the disc space. For example, the two components could be connected with a screw 2206. The assembled cage is too large to extrude through the AF defect used to insert the components separately.

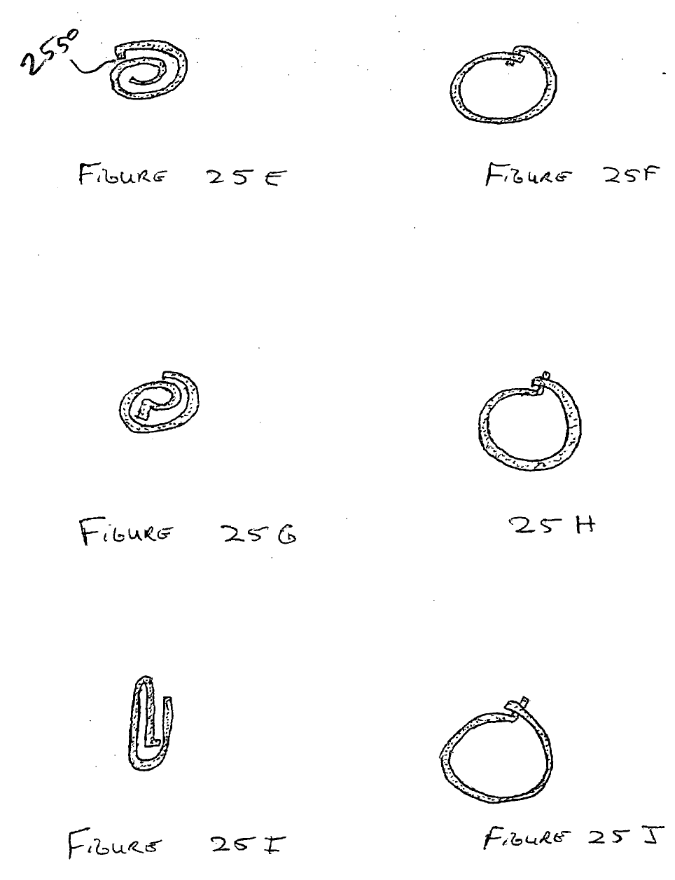

FIG. 23A is an axial cross-section of an exploded, alternative embodiment of a NR device according to the invention. The enclosure of the device encloses the cushion material, and a hole in the enclosure provides a path to insert a screw. FIG. 23B is an axial cross-section of an assembled embodiment of the device drawn in FIG. 23A. FIG. 24A is a view of the top of an alternative embodiment of a stiff component. Rigid cylinder shaped bead-like components are threaded over a cable. The cable may have elastic properties. The component is folded and placed into the NR enclosure.

FIG. 24B is a view of the top the component drawn in FIG. 24A. The device is drawn in its extended shape. The device assumes its extended shape after it is placed into the enclosure. FIG. 24C is an oblique view of a cap component. The cap component is placed over a portion of both bead-like components. The cap stiffens the components by preventing the cable from bending between the bead-like components.

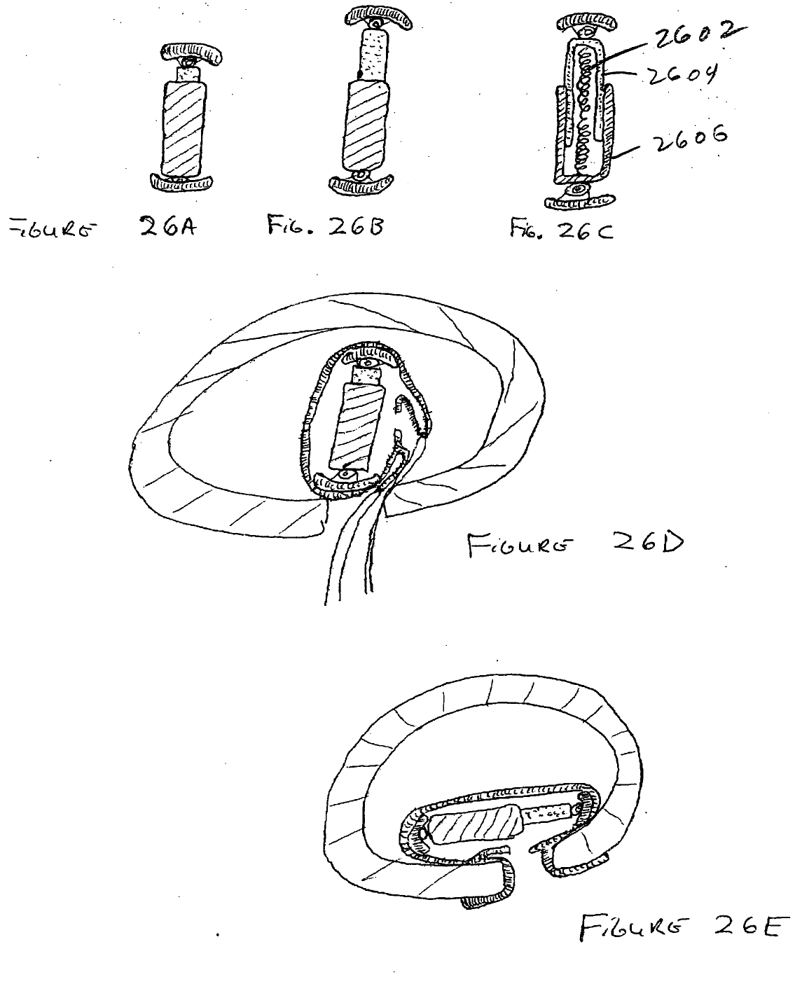

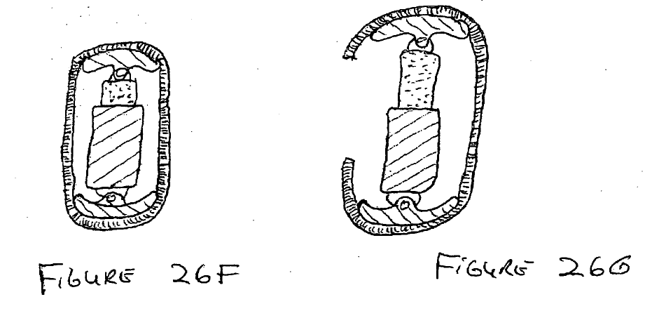

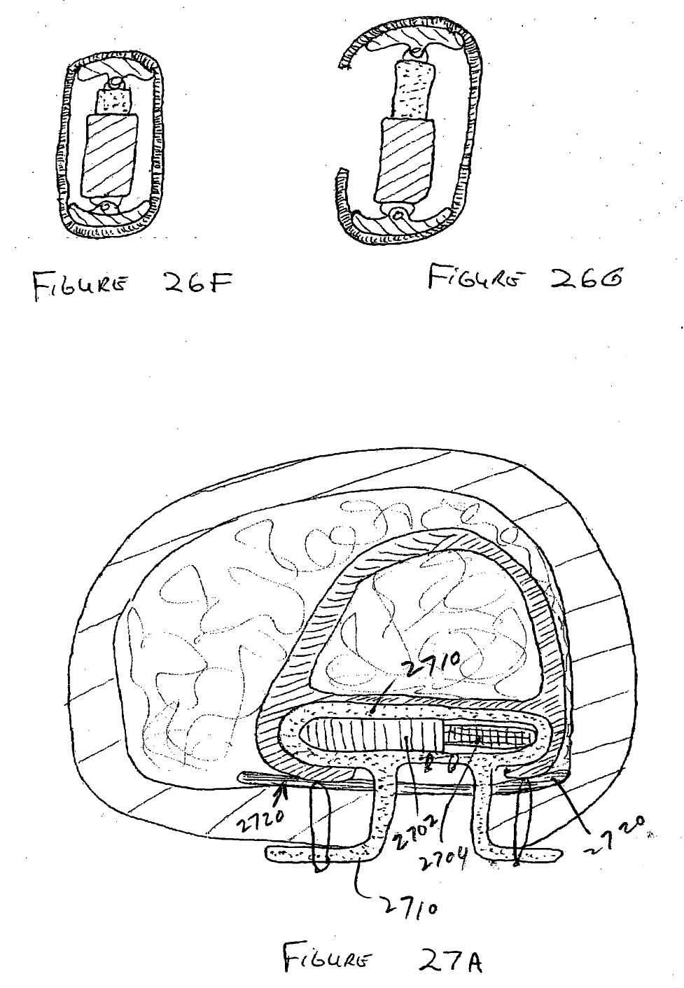

FIG. 24D is a lateral view of the device drawn in FIG. 24A. The cap of FIG. 24C is drawn in position on the bead-like components. FIG. 25A is a view of the top of an alternative embodiment of the stiff component. The component is a spring or made of a shape memory material. The device is drawn in its first, or compressed shape.

FIG. 25B is a view of the top of the embodiment of the stiff component drawn in FIG. 25A. The component is drawn in its second, or expanded shape. The component assumes its second shape after placement in the disc space. The shape change could occur as pressure is released from the component or as a reaction to temperature change, etc. The component has a locking mechanism to hold the expanded shape. For example, projections 2510 from one end of the component could cooperate with the other end of the component to form the lock.

FIG. 25C is a view of the top of an alternative embodiment of the component drawn in FIG. 25A. The locking components project from the inner surface of the coiled device. FIG. 25D is a view of the top of the embodiment of the device drawn in FIG. 25C. The device is drawn in its locked, expanded shape. Alternatively, teeth-like projections could project from both ends of the device. The teeth could interdigitate to lock the device in its expanded position.

FIG. 25E is a view of the top of an alternative embodiment of the stiff component drawn in FIG. 25C. The device is drawn in its coiled, first shape. FIG. 25F is a view of the top of the embodiment of the device drawn in FIG. 25E. The device is drawn in its expanded shape. A projection 2550 from one end of the device fits into a slot on the other end of the device to lock the component in its expanded shape.

FIG. 25G is a view of the top of an alternative embodiment of the stiff component drawn in FIG. 25E. The device is drawn in its coiled first shape. FIG. 25H is a view of the top of the embodiment of the device drawn in FIG. 25G. The device is drawn locked in its expanded position. FIG. 251 is a view of the top of an alternative embodiment of the device drawn in FIG. 25G. The device is oval or oblong in shape. The narrow device fits through smaller windows in the AF.

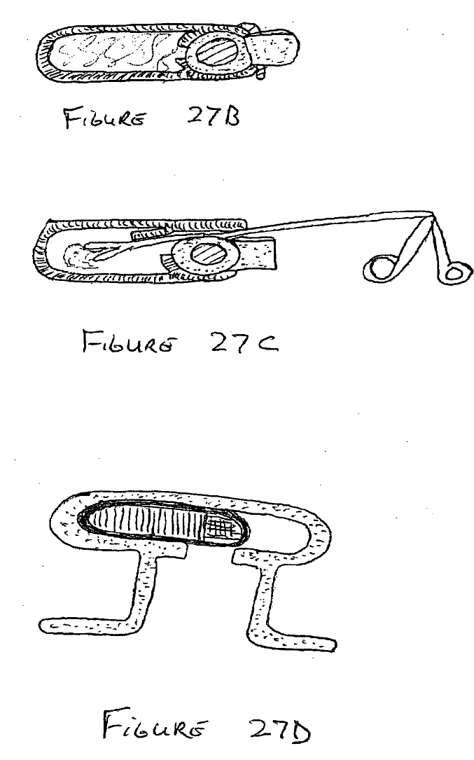

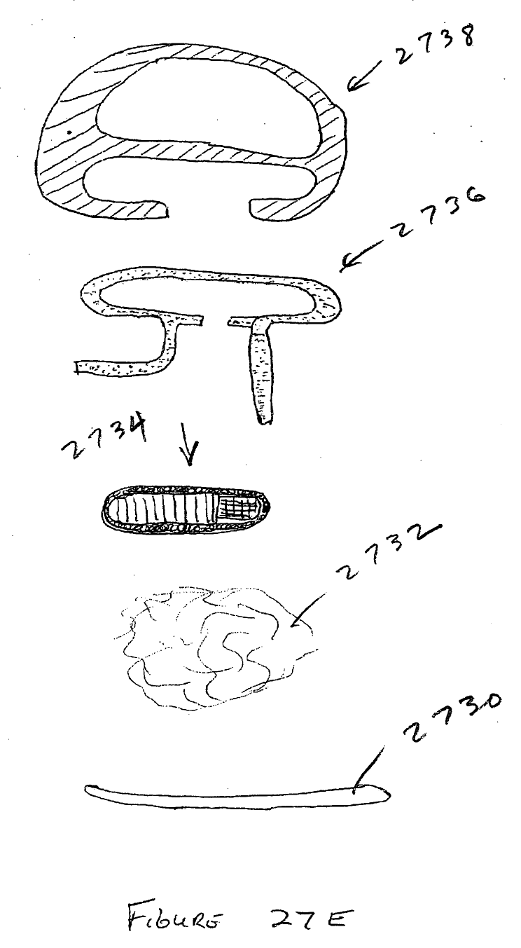

FIG. 25J is a view of the top of the device drawn in FIG. 251. The device is drawn locked in its expanded shape. The stiff component and/or the enclosure could be made of an absorbable material. FIG. 26A is a lateral view of another embodiment of the stiff component. The component is drawn in its compressed position. The curved pieces on the top and the bottom of the device area connected to two spring loaded cylinders. The curved pieces may be connected to the cylinders with axles that allow the components to swivel.

FIG. 26B is a lateral view of the stiff device drawn in FIG. 26A. The device is drawn in its extended position. A spring 2602 that lies within the two cylinders 2604, 2606 forces the springs apart as compression is released from the device. FIG. 26C is a cross-section of the device drawn in FIG. 26B.

FIG. 26D is an axial cross-section of the disc, an enclosure, and the stiff device drawn in FIG. 26B. The stiff device has been placed into the enclosure device before placing the two components into the disc. The opening in the enclosure is seen on the right side of the drawing. Strings are attached to the two flaps of the enclosure. The stiff device is held in its compressed shape by a band that surrounds the stiff device. The band was not drawn around the device to better illustrate the stiff device and the enclosure. The strings can be used to rotate the enclosure and the stiff device within the enclosure 90 degrees.

FIG. 26E is an axial cross-section of the disc, the enclosure, and the embodiment of the stiff device drawn in FIG. 26D. The enclosure and the stiff device within the enclosure have been rotated 90 degrees from the orientation of the device drawn in FIG. 26D. The stiff device is drawn in its extended position. The enclosure is filled with a filler material after positioning the device with in the disc space. The enclosure may be attached to the AF as illustrated in FIG. 16H.

FIG. 26F is a lateral view of the stiff component drawn in FIG. 26A and compression band. The compression band surrounds the stiff device to maintain the device in its compressed position. FIG. 26G is a lateral view of the stiff component drawn in FIG. 26F and the compression band. The compression band has been cut. The stiff device expands after cutting the compression device. The compression band is cut after the stiff device is inserted into the disc space and after the device has been rotated 90 degrees.

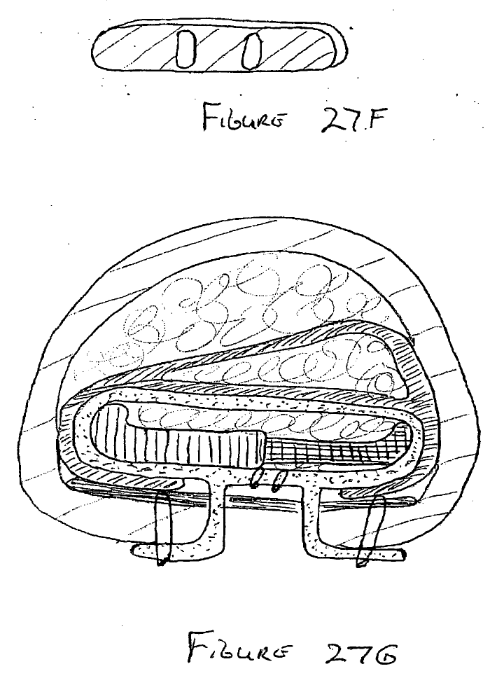

FIG. 27A is an axial cross-section and an alternative embodiment of the device drawn in FIG. 26E. The spring loaded stiff component is similar to the component drawn in FIG. 26A. The pieces that swivel on the ends of the component in FIG. 26E are not included in this embodiment of the device. The enclosure has two compartments. One compartment contains only cushion, filler material. The second compartment contains the stiff component. The second compartment may also contain cushion, filler, material. The enclosure (area of the drawing with closely spaced diagonal lines) is made of an elastic material such as, but not limited to, polyolefin copolymers, polyethylene polycarbonate, polyethylene terephthalate. The enclosure may have small holes to allow fluids to pass in and out of the device. The elastic enclosure transmits loads to the retained NP and the AF.

The stiff device 2702, 2704 is surrounded by a reinforced material 2710. The reinforced material may be incorporated in the portion of the enclosure that surrounds the stiff component. The reinforced material also forms the flaps that may be sewn to the AF. The reinforced material includes, but is not limited to, Dacron and woven high molecular weight polyethylene polyester. A dam, cover component 2720 is drawn between the device and the inner surface of the AF. The flaps of the reinforced material extend through holes in the cover component. The flaps of the device have been sewn or stapled to the AF. The device may also be attached to the vertebrae. The vertebrae may be osteotomized to insert the device as taught in my co-pending application International Patent Application PCT/US03/12755.

This embodiment of the device selectively transfers loads to the anterior and lateral AF as taught in my co-pending application U.S. patent application Ser. No. 10/407,554. The compartment in the device that contains the stiff component prevents the stiff component from rotating away from the AF after the device is placed. Attaching the reinforced material to the AF or the vertebrae also help hold the stiff component in place. The cover between the device and the AF helps prevent filler material from extruding from the device. The stiff component is drawn in its expanded position. The areas of the drawing with wavy lines represent the retained NP and the filler cushion material. As noted in the other embodiments of the invention the filler material could be a natural or synthetic substance. The stiff component could also be incorporated into the posterior wall or the posterior and lateral walls of the enclosure. The device can be inserted as depicted in FIGS. 26D and 26E.

FIG. 27B is a sagittal cross-section of the device drawn in FIG. 27A. The reinforced material is represented by the dotted area of the drawing. The area of the drawing with widely spaced diagonal lines represents the stiff component. The enclosure is represented by the area of the drawing with closely spaced diagonal lines. The walls of the enclosure that surround the stiff component have an opening to insert the filler material.

FIG. 27C is a sagittal cross-section of the device drawn in FIG. 27B. The figure shows how the device may be filled. An instrument can be inserted between the enclosure and the stiff device. The walls of the enclosure act as flap valves when the instrument is withdrawn from the device. The flap valves help prevent filler material from extruding from the device.

FIG. 27D is cross-section of the reinforced material around the embodiment of the stiff device drawn in FIG. 27A and the stiff device. The stiff device is drawn in its compressed position. The dark line around the stiff component represents a tension band. The tension band is incised after the device is inserted and rotated within the disc space.

FIG. 27E is an exploded partial cross-section of the embodiment of the device drawn in FIG. 27A. The oval 2730 at the bottom of the drawing represents the cover component. The area of the drawing 2732 represents the filler material. The compressed stiff device is shown at 2734, and the reinforced material that surrounds the stiff device is depicted at 2736. One of the flaps of the reinforced material is drawn in a position that aids treading the flaps through the holes in the cover component. The enclosure is indicated at 2738.

FIG. 27F is an oblique view of the optional cover component drawn in FIG. 27A. FIG. 27G is a partial axial cross-section of the disc and an alternative embodiment of the invention drawn in FIG. 27A. The stiff component extends across the entire posterior portion of the AF and a portion of both lateral walls of the AF. Filler material is drawn within both compartments of the enclosure.

I claim:

1. A nucleus replacement (NR) device, comprising:
a fillable enclosure configured to consume some or all of an intradiscal space; and
a composite cushioning material disposed within the enclosure to produce asymmetrical stiffness of the device.

2. The NR of claim 1, wherein the cushioning material increases the lateral stiffness of the device.

3. The NR of claim 1, further including a stiffer or higher durometer component disposed within the enclosure.

4. The NR of claim 1, further including a stiffer or higher durometer component oriented laterally within the enclosure when the device is positioned within an intradiscal space.

5. The NR of claim 1, further including a stiffer or higher durometer component that extends through the enclosure.

6. The NR of claim 1, further including a plurality of stiffer or higher durometer components disposed within the enclosure.

7. The NR of claim 1, further including a plurality of intersecting stiffer or higher durometer components disposed within the enclosure.

8. The NR of claim 1, further including a plurality of transversely oriented stiffer or higher durometer components disposed within the enclosure.

9. The NR of claim 1, further including one or more stiffer or higher durometer components that are assembled in the disc space.

10. The NR of claim 1, further including a stiffer or higher durometer component with a spring or shape-memory material that becomes wider after the NR is placed in an intradiscal space.

11. The NR of claim 1, further including a stiffer or higher durometer component with a spring or shape-memory material that becomes laterally wider after the NR is placed in an intradiscal space.

12. The NR of claim 1, further including one or more stiffer arms that rotate from a narrower first position to a wider second position.

13. The NR of claim 1, further including a stiffer hoop or band and a component that extends across the band.

14. The NR of claim 1, further including a stiffer or higher durometer component disposed within the enclosure; and
wherein one or more of the enclosure, cushioning material, or component is assembled within an intradiscal space.

15. The NR of claim 1, wherein the enclosure is flexible.

16. The NR of claim 1, wherein one component of the cushioning material exhibits an increased durometer following an in-situ curing process.

17. The NR of claim 16, wherein the material that exhibits an increased durometer is an in-situ forming polymer.

18. The NR of claim 16, wherein the material that exhibits an increased durometer includes a liquid metal.

19. The NR of claim 1, further including a stiffer or higher durometer component disposed within the enclosure that changes size or shape when the device is positioned within an intradiscal space.

20. The NR of claim 1, further including a stiffer or higher durometer component disposed within the enclosure;
wherein the enclosure and/or the component are absorbable.
